(19) United States
(12) Patent Application Publication
(10) Pub. No.: US 2026/0014317 A1
(43) Pub. Date: Jan. 15, 2026

(54) SUBCUTANEOUS INFUSION CONTROL SYSTEM

(71) Applicant: CareFusion 303, Inc., San Diego, CA (US)

(72) Inventor: Adel SHAMS, Verona, NJ (US)

(21) Appl. No.: 18/770,421

(22) Filed: Jul. 11, 2024

(51) Int. Cl. A61M 5/172

(52) U.S. Cl. CPC A61M 5/1723; A61M 2205/18; A61M 2230/65

(57) ABSTRACT

Control systems for infusions are described herein. An infusion control system includes a sensor and a controller. The sensor is configured to measure a subcutaneous fluid level. The control is configured to monitor the subcutaneous fluid level during a subcutaneous fluid infusion provided by an infusion system. Further the controller is configured to provide a feedback signal if the measured subcutaneous fluid level exceeds a subcutaneous fluid level threshold.

### FIELD OF THE INVENTION

The present disclosure generally relates to infusion systems, and in particular, to control of infusion systems.

### BACKGROUND

Medical treatments often include the subcutaneous infusion of a medical fluid to patients using infusion components that are connected though an arrangement of flexible tubing and fittings to a source of fluid. Subcutaneous infusion of medical fluids can be used for various purposes, such as hydration, pain management, and immunoglobulin (SCIg) therapies. Subcutaneous infusions can be relatively easy and inexpensive to administer.

Certain conventional delivery mechanisms for subcutaneous infusions deliver the medical fluid at a fixed rate or constant pressure. During the infusion process, the absorption of the medical fluid is determined by the diffusion of the medical fluid into the hypodermis tissue, passage through the vessel wall, and capillary blood perfusion. In some applications, it is desired to simplify the effective administration of subcutaneous infusions while avoiding complications.

### SUMMARY

The disclosed subject matter relates to control of fluid infusions. In certain embodiments, infusion control systems are disclosed that comprise a sensor configured to measure a subcutaneous fluid level; and a controller configured to: monitor the subcutaneous fluid level during a subcutaneous fluid infusion provided by an infusion system; and provide a feedback signal if the measured subcutaneous fluid level exceeds a subcutaneous fluid level threshold.

In certain embodiments, a method to control an infusion system is disclosed that comprises providing a medical fluid to subcutaneous tissue of a patient via an infusion system; measuring a fluid level of the subcutaneous tissue via a sensor; comparing the measured fluid level to a fluid level threshold via a controller; and providing a feedback signal via the controller if the fluid level exceeds the fluid level threshold.

In certain embodiments, an infusion system is disclosed that comprises an infusion pump configured to provide medical fluid to subcutaneous tissue of a patient; a sensor configured to measure a fluid level of the subcutaneous tissue of the patient; and a controller configured to: monitor the fluid level of the subcutaneous tissue during a subcutaneous fluid infusion provided by the infusion pump; and adjust an infusion rate of the infusion pump if the measured fluid level of the subcutaneous tissue exceeds a subcutaneous fluid level threshold.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 depicts a patient receiving an infusion of a medical fluid using an IV pump.

FIG. 2 depicts subcutaneous tissue of a patient receiving a subcutaneous infusion.

FIG. 3 depicts a flow chart of the control of the infusion system.

### DETAILED DESCRIPTION

Embodiments of the disclosed infusion control system provide closed loop control of the subcutaneous infusion process. The infusion control system includes one more sensors to detect fluid levels within the subcutaneous tissue and a controller to adjust the infusion rate to maximize medical fluid delivery while avoid complications such as edema.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed control systems for the subcutaneous infusion or administration of medical fluid, it is to be understood that this description is only an example of usage and does not limit the scope of the claims. Various aspects of the disclosed control system may be used in any application where it is desirable to provide closed loop control of the administration or infusion of medical fluids.

The disclosed control system overcomes several challenges discovered with respect to certain conventional subcutaneous infusion or delivery systems and/or processes. One challenge with certain conventional subcutaneous infusions, such as large volume subcutaneous infusions, is that infusions can results in excess medical fluid in the subcutaneous tissue, overwhelming the body’s ability to absorb the fluid. In certain conventional applications, excess medical fluid can accumulate if the rate of fluid being administered is too high or if an improper infusion technique is utilized. If subcutaneous medication is not systemically absorbed by the capillaries in the subcutaneous tissue after penetrating the extracellular matrix, excess medical fluid can accumulate in the tissues surrounding the infusion site, leading to a local edema. Accumulated or excess medical fluid in the subcutaneous tissue can lead to swelling, pain, and/or discomfort at the infusion site. Because certain conventional delivery mechanisms for subcutaneous infusions may deliver medical fluid at a fixed rate or constant pressure regardless of the fluid level within the subcutaneous fluid level, the use of certain conventional delivery mechanisms is undesirable for subcutaneous infusions.

Therefore, in certain conventional applications, a clinician may visually monitor the infusion site, adjust the infusion site (e.g., reposition the needle or catheter, change the needle length, needle gauge, and/or injection site), apply warm compresses, and/or adjust the infusion rate to prevent or manage local edema during subcutaneous infusion. Further, in certain conventional applications, a clinician may utilize multiple infusion sites to prevent or manage local edema. Because certain conventional delivery mechanisms for subcutaneous infusions may require frequent and skilled intervention to prevent or manage local edema during subcutaneous infusion, the use of certain conventional delivery mechanisms is undesirable for subcutaneous infusions. Further, since frequent and skilled intervention may be difficult to coordinate in non-acute settings, such as home hospice, the use of certain conventional delivery mechanisms is undesirable for subcutaneous infusions in these settings.

Therefore, in accordance with the present disclosure, it is advantageous to provide a control system for subcutaneous infusions that allows for real-time monitoring of subcutaneous tissue fluid levels. Further, it is advantageous to provide closed-loop control and dynamic adjustment of the subcutaneous infusion rate based on the fluid levels within the subcutaneous tissue. Additionally, embodiments described in the present disclosure allow for enhanced therapeutic precision, minimizing the risk of under-dosing and/or overdosing. Further, embodiments described in the present disclosure allow for clinicians to remotely monitor, administer medical fluids and/or confirm the of positioning of the subcutaneous infusion site. Also, embodiments described in the present disclosure provide automated control of the infusion process to minimize patient intervention and increase patient compliance.

Examples of control systems for infusion systems and closed loop infusion systems that allow for real-time monitoring and/or closed-loop subcutaneous infusion to allow for fluid administration while minimizing complications are now described.

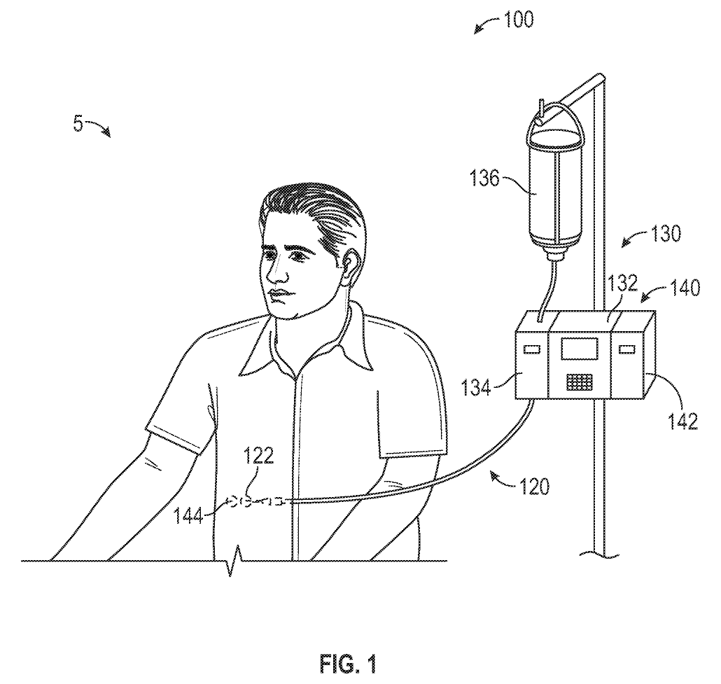

FIG. 1 illustrates a patient 5 receiving a subcutaneous infusion of a medical fluid (e.g., hydration, pain management, and immunoglobulin (SCIg) therapies) through an infusion system 100 according to certain aspects of the present disclosure. In the depicted example, a delivery system 130 delivers medical fluid to the patient 5. As illustrated, the delivery system 130 includes a pump module 134 to deliver medical fluid stored in a container 136 to the patient 5. Medical fluid flows between the container 136 and the patient 5 via a tubing set 120. Prior to operation, components of the tubing set 120 can be primed with medical fluid.

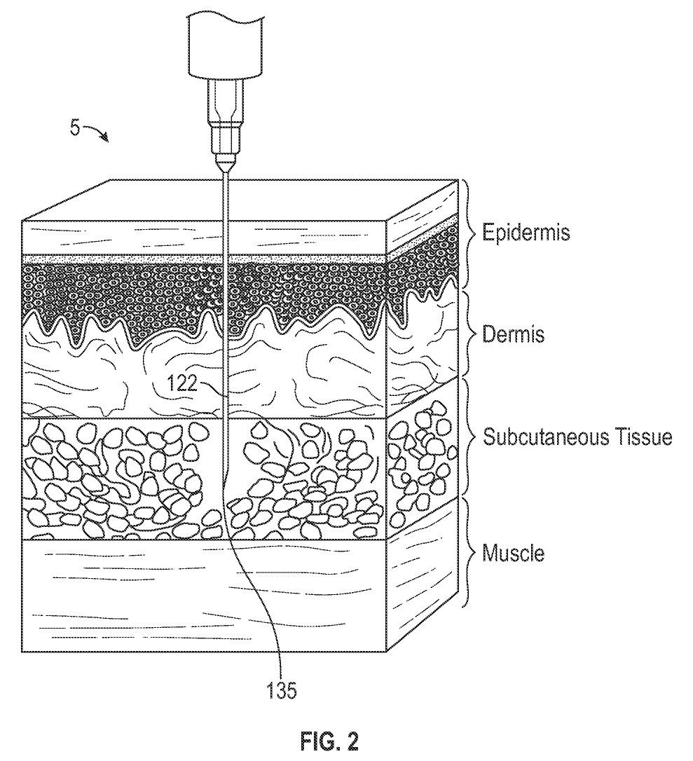

FIG. 2 depicts subcutaneous tissue of a patient 5 receiving a subcutaneous infusion. With reference to FIGS. 1 and 2, the infusion system 100 delivers medical fluid 135 to subcutaneous tissue (depicted in FIG. 2) via one or more subcutaneous needles 122 positioned within the subcutaneous tissue and connected to the tubing set 120. As illustrated, medical fluid 135 can be pumped from the container 136 and through the tubing set 120 to the subcutaneous needle 122 via the pump module 134. In the depicted example, subcutaneous infusion of medical fluid 135 can be systemically absorbed by the blood vessels and capillaries in the subcutaneous tissue after penetrating the extracellular matrix. Subcutaneous infusion of medical fluid 135 can be used for various purposes, such as hydration, pain management, and immunoglobulin (SCIg) therapies. Advantageously, subcutaneous infusion of medical fluid can be easier and more inexpensive to administer compared to intravenous (IV) infusions. In some applications, subcutaneous infusions can be used for large volume subcutaneous infusions of medical fluid 135. In the depicted example, the delivery system 130 includes a pump controller 132 to control the operation of pump module 134 and the delivery of medical fluid 135 from the container 136 to the patient 5.

In the depicted example, the infusion system 100 includes a subcutaneous infusion control system 140 to monitor and/or control the flow of medical fluid 135 into the subcutaneous tissue of the patient 5. In some embodiments, the subcutaneous infusion control system 140 can monitor fluid levels within the subcutaneous tissue of the patient 5 and provide feedback to a clinician. In some embodiments, the subcutaneous infusion control system 140 can provide closed loop control of the delivery system 130 by adjusting the operation of the delivery system 130 in response to fluid levels within the subcutaneous tissue of the patient 5. In some embodiments, the subcutaneous infusion control system 140 can be integrated with the delivery system 130 or the pump controller 132. In some embodiments, components of the subcutaneous infusion control system 140 can be separate from the delivery system 130 or the pump controller 132. Advantageously, monitoring and/or closed loop control of the delivery system 130 based on fluid levels within the subcutaneous tissue of the patient 5 can prevent, minimize, and/or manage complications such as edema, while allowing for precise control of dosing, and remote monitoring or control of the infusion system 100.

In the depicted example, the subcutaneous infusion control system 140 includes one or more fluid level sensors 144 to detect or measure fluid levels within the subcutaneous tissue of the patient 5. One or more fluid level sensors 144 can be utilized for each infusion site. During operation, the fluid level sensors 144 transmit subcutaneous fluid level data to the subcutaneous infusion controller 142 either wired or wirelessly. In some applications, the fluid level sensors 144 can provide fluid data, before, during, and/or after a subcutaneous infusion. As described herein, fluid data from the fluid level sensors 144 can allow for real-time monitoring and/or control of the delivery system 130 to minimize complications and allow for enhanced drug delivery control.

As illustrated, the fluid level sensors 144 can be disposed on the skin of the patient 5 near the infusion site of the subcutaneous needle 122 to detect fluid levels within the subcutaneous tissue. In some embodiments, the fluid level sensors 144 can be worn on the body of the patient 5. In some embodiments, the fluid level sensors 144 can include a portion that goes beneath the surface of the skin of the patient 5.

In some embodiments, the fluid level sensors 144 are bioimpedance fluid sensors. For example, bioimpedance fluid sensors can measure local extracellular matrix fluid levels (e.g., utilizing low frequency signals) and local intracellular matrix fluid levels (e.g., utilizing high frequency signals) in proximity to the injection site. In some embodiments, other types of fluid level sensors 144 can be implemented for use with the subcutaneous infusion control system 140.

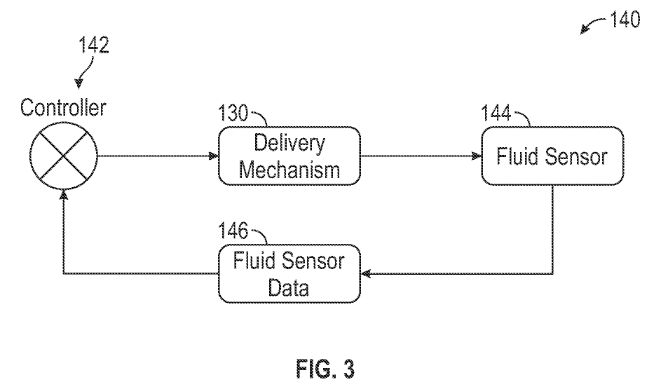

FIG. 3 depicts a flow chart of the control of the infusion system 100. With reference to FIGS. 1 and 3, the subcutaneous infusion control system 140 include a subcutaneous infusion controller 142 to monitor fluid levels or accumulation within the subcutaneous tissue using fluid sensor data 146 from the fluid level sensors 144. As described herein, subcutaneous fluid sensor data 146 is provided to the subcutaneous infusion controller 142 from the fluid level sensors 144. The subcutaneous infusion controller 142 can process fluid sensor data 146 from the fluid level sensors 144 to monitor the condition of the patient 5 and the infusion process. In the depicted example, the subcutaneous infusion controller 142 can process fluid sensor data 146 from the fluid level sensors 144 in real-time.

In some embodiments, the subcutaneous infusion controller 142 can use one or more algorithms to analyze the fluid sensor data 146. In the depicted example, the subcutaneous infusion controller 142 compares the fluid sensor data 146 to one or more fluid level thresholds. The fluid level thresholds can represent the maximum allowable fluid level or accumulation within the subcutaneous infusion site to avoid edema, pain, swelling, and/or patient discomfort. In some applications, the fluid level thresholds can be predetermined, calculated from patient data, medical fluid properties, historical data, and/or calculated using one or more algorithms.

In some embodiments, the subcutaneous infusion controller 142 calculates the rate of change of the fluid level within the monitored subcutaneous tissue based on the fluid sensor data 146. The subcutaneous infusion controller 142 can compare calculated rate of change of the subcutaneous fluid level to a rate of change threshold. Similarly, the rate of change fluid level thresholds can represent the maximum allowable rate of change of fluid within the subcutaneous infusion site to avoid edema, pain, swelling, and/or patient discomfort. In some applications, the rate of change fluid level thresholds can be predetermined, calculated from patient data, medical fluid properties, historical data, and/or calculated using one or more algorithms. In some embodiments the subcutaneous infusion controller 142 can compare both the fluid level and the rate of change of the fluid level within the subcutaneous tissue against their respective thresholds.

In some embodiments, the rate of change thresholds can be based on the prescribed or predetermined infusion rates for various types of medical fluids. For example, immunoglobulin (SCIg) therapies with higher infusion rates (e.g., 20-300 ml/min) may have a higher rate of change fluid level threshold than hydration infusions (e.g., 250 mL/h) or pain or antibiotic infusions (e.g., < 5mL/h). In some applications, the rate of change threshold may be lower than the infusion rate of the medical fluid to account for absorption in the subcutaneous tissue. Further, in some embodiments, the rate of change thresholds may be calculated or selected to avoid rapid spikes in fluid levels (e.g., rapid changes in excess of 5% or 10% of a base fluid index), while accounting for, or otherwise tolerating long term changes in a patient’s hydration (e.g., changes over the course of 12 to 24 hours in the patient’s hydration index).

During operation, if the fluid level and/or the rate of change of the fluid level exceeds the selected threshold, the subcutaneous infusion controller 142 can provide a feedback signal that the threshold has been exceeded.

In some embodiments, the feedback signal can be provided to a clinician. In some applications, the feedback signal can be a local signal (e.g., an auditory or visual signal) or a remote signal or alert provided to a clinician or other monitor. In response to the feedback signal, a clinician may manually monitor or intervene by visually monitoring the infusion site, adjusting the infusion site (e.g., repositioning the needle or catheter, changing the needle length, needle gauge, and/or injection site), applying warm compresses, and/or adjusting the infusion rate to prevent or manage local edema during the subcutaneous infusion.

In some embodiments, the feedback signal from the subcutaneous infusion controller 142 can be provided to the delivery system 130 to allow for closed loop control of the delivery system 130. In other words, in some embodiments, the subcutaneous infusion controller 142 can utilize the fluid sensor data 146 from the fluid level sensor 144 to adjust the flow rate of the infusion to the subcutaneous tissue of the patient 5. In the depicted example, the feedback signal from the subcutaneous infusion controller 142 can include instructions to adjust the infusion or flow rate from the pump module 134 to the subcutaneous tissue of the patient 5. In some embodiments, the feedback signal from subcutaneous infusion controller 142 is provided to the pump controller 132 to instruct the pump module 134 to adjust the flow rate of the pump module 134.

In some applications, the subcutaneous infusion controller 142 can utilize one or more algorithms to analyze the fluid sensor data 146 and adjust the drug delivery rate of the delivery system 130. In some embodiments, the subcutaneous infusion controller 142 may adjust the drug delivery rate of the delivery system 130 in proportion to the difference between the current fluid level and the desired fluid level, or the current rate of change of the fluid level and the desired rate of change of the fluid level. For example, in some embodiments, the subcutaneous infusion controller 142 may utilize a proportional–integral–derivative controller to adjust the drug delivery rate of the delivery system 130 based on the fluid sensor data 146.

Advantageously, closed-loop control of the delivery system 130 allows for enhanced therapeutic precision and dosing, while enabling remote monitoring and administration of subcutaneous infusions. In some embodiments, the subcutaneous infusion controller 142 can be a separate component from the pump controller 132 and/or pump module 134. In some embodiments, the subcutaneous infusion controller 142 can be integrated with the components of the delivery system 130, such as the pump controller 132 and/or pump module 134.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. An infusion control system, comprising: a sensor configured to measure a subcutaneous fluid level; and a controller configured to: monitor the subcutaneous fluid level during a subcutaneous fluid infusion provided by an infusion system; and provide a feedback signal if the measured subcutaneous fluid level exceeds a subcutaneous fluid level threshold.

Clause 2. The infusion control system of Clause 1, wherein the sensor comprises a bioimpedance sensor.

Clause 3. The infusion control system of Clause 1, wherein the sensor is configured to measure an extracellular fluid level.

Clause 4. The infusion control system of Clause 1, wherein the sensor is configured to measure an intracellular fluid level.

Clause 5. The infusion control system of Clause 1, wherein the controller is further configured to: monitor a rate of change of the subcutaneous fluid level during the subcutaneous fluid infusion; and provide the feedback signal if the rate of change of the subcutaneous fluid level exceeds a rate of change threshold.

Clause 6. The infusion control system of Clause 5, wherein the controller comprises a proportional–integral–derivative controller.

Clause 7. The infusion control system of Clause 5, wherein the rate of change threshold corresponds to an edema condition.

Clause 8. The infusion control system of Clause 1, wherein the subcutaneous fluid level threshold corresponds to an edema condition.

Clause 9. The infusion control system of Clause 1, wherein the controller is configured to provide the feedback signal to a clinician.

Clause 10. The infusion control system of Clause 1, wherein the controller is configured to provide the feedback signal to an infusion system.

Clause 11. The infusion control system of Clause 10, wherein the feedback signal includes an instruction to adjust an infusion rate.

Clause 12. A method to control an infusion system, the method comprising: providing a medical fluid to subcutaneous tissue of a patient via an infusion system; measuring a fluid level of the subcutaneous tissue via a sensor; comparing the measured fluid level to a fluid level threshold via a controller; and providing a feedback signal via the controller if the fluid level exceeds the fluid level threshold.

Clause 13. The method of Clause 12, wherein the fluid level threshold corresponds to an edema condition.

Clause 14. The method of Clause 12, further comprising: calculating a rate of change of the fluid level during the providing of the medical fluid; comparing the rate of change of the fluid level to a rate of change threshold via a controller; and providing a feedback signal via the controller if the rate of change exceeds the rate of change threshold.

Clause 15. The method of Clause 14, wherein the fluid level threshold corresponds to an edema condition.

Clause 16. The method of Clause 12, further comprising: instructing the infusion system to reduce an infusion rate via the feedback signal.

Clause 17. An infusion system, comprising: an infusion pump configured to provide medical fluid to subcutaneous tissue of a patient; a sensor configured to measure a fluid level of the subcutaneous tissue of the patient; and a controller configured to: monitor the fluid level of the subcutaneous tissue during a subcutaneous fluid infusion provided by the infusion pump; and

adjust an infusion rate of the infusion pump if the measured fluid level of the subcutaneous tissue exceeds a subcutaneous fluid level threshold.

Clause 18. The infusion system of Clause 17, wherein the controller is integrated with the infusion pump.

Clause 19. The infusion system of Clause 17, wherein the controller is separate from the infusion pump.

Clause 20. The infusion system of Clause 17, wherein the sensor comprises a bioimpedance sensor.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. §101, 102, or 103, nor should they be interpreted in such a way.

What is claimed is:

1. An infusion control system, comprising:
a sensor configured to measure a subcutaneous fluid level; and
a controller configured to:
monitor the subcutaneous fluid level during a subcutaneous fluid infusion provided by an infusion system; and
provide a feedback signal if the measured subcutaneous fluid level exceeds a subcutaneous fluid level threshold.

2. The infusion control system of claim 1, wherein the sensor comprises a bioimpedance sensor.

3. The infusion control system of claim 1, wherein the sensor is configured to measure an extracellular fluid level.

4. The infusion control system of claim 1, wherein the sensor is configured to measure an intracellular fluid level.

5. The infusion control system of claim 1, wherein the controller is further configured to:
monitor a rate of change of the subcutaneous fluid level during the subcutaneous fluid infusion; and
provide the feedback signal if the rate of change of the subcutaneous fluid level exceeds a rate of change threshold.

6. The infusion control system of claim 5, wherein the controller comprises a proportional–integral–derivative controller.

7. The infusion control system of claim 5, wherein the rate of change threshold corresponds to an edema condition.

8. The infusion control system of claim 1, wherein the subcutaneous fluid level threshold corresponds to an edema condition.

9. The infusion control system of claim 1, wherein the controller is configured to provide the feedback signal to a clinician.

10. The infusion control system of claim 1, wherein the controller is configured to provide the feedback signal to an infusion system.

11. The infusion control system of claim 10, wherein the feedback signal includes an instruction to adjust an infusion rate.

12. A method to control an infusion system, the method comprising:
providing a medical fluid to subcutaneous tissue of a patient via an infusion system;
measuring a fluid level of the subcutaneous tissue via a sensor;
comparing the measured fluid level to a fluid level threshold via a controller; and
providing a feedback signal via the controller if the fluid level exceeds the fluid level threshold.

13. The method of claim 12, wherein the fluid level threshold corresponds to an edema condition.

14. The method of claim 12, further comprising:
calculating a rate of change of the fluid level during the providing of the medical fluid;
comparing the rate of change of the fluid level to a rate of change threshold via a controller; and
providing a feedback signal via the controller if the rate of change exceeds the rate of change threshold.

15. The method of claim 14, wherein the fluid level threshold corresponds to an edema condition.

16. The method of claim 12, further comprising:
instructing the infusion system to reduce an infusion rate via the feedback signal.

17. An infusion system, comprising:
an infusion pump configured to provide medical fluid to subcutaneous tissue of a patient;
a sensor configured to measure a fluid level of the subcutaneous tissue of the patient; and
a controller configured to:
monitor the fluid level of the subcutaneous tissue during a subcutaneous fluid infusion provided by the infusion pump; and
adjust an infusion rate of the infusion pump if the measured fluid level of the subcutaneous tissue exceeds a subcutaneous fluid level threshold.

18. The infusion system of claim 17, wherein the controller is integrated with the infusion pump.

19. The infusion system of claim 17, wherein the controller is separate from the infusion pump.

20. The infusion system of claim 17, wherein the sensor comprises a bioimpedance sensor.
